(19) United States
(12) Patent Application Publication
(10) Pub. No.: US 2003/0124160 A1
(43) Pub. Date: Jul. 3, 2003

(54) Targeted drug delivery

(76) Inventors: Stefana M. Petrescu, Bucharest (RO); Gertrude E. Costin, Bucharest (RO); Mihaela Trif, Bucharest (RO); Norica Nichita, Bucharest (RO); Raymond A. Dwek, Oxford (GB)

Correspondence Address: FOLEY AND LARDNER, SUITE 500, 3000 K STREET NW, WASHINGTON, DC, 20007, US

(21) Appl. No.: 10/269,852

(22) Filed: Oct. 11, 2002

Related U.S. Application Data
(60) Provisional application No. 60/328,426, filed on Oct. 12, 2001.

(51) Int. Cl. A61K 9/127; A61K 7/00

(52) U.S. Cl. 424/401; 424/450

(57) ABSTRACT

The use of pH-sensitive liposomes enhances the delivery of drugs that target the endoplasmic reticulum, reducing the required dosage compared to direct administration of the drug without such liposomes. In particular, antiviral compounds such as N-butyldeoxynojirnmycin can be used in lower amounts when administered in a pH-sensitive liposome.

### FIELD OF THE INVENTION

The present invention relates to delivery of drugs, particularly antiviral drugs, across the cell membrane using pH-sensitive liposomes.

### BACKGROUND OF THE INVENTION

The use of certain pH-sensitive liposomes to deliver molecules into the cytosol has been described. For example, Lee et al. (J. Biol. Chem., 271(13): 7249-7252 (1996)) disclosed that certain pH-sensitive liposomes could be used to deliver hemolysin or fluorescent dye into the cytosol.

Straubinger (Methods In Enzymology, 221(1993): 361-376) reviews results of various studies with pH-sensitive liposomes. This article includes a discussion of how to formulate various pH-sensitive liposomes.

Daemen et al. (Hepatology, 26(2): 416-423 (1997)) demonstrated that certain pH-insensitive liposomes delivered gold colloidal particles at relatively higher amounts to the liver and lower amounts to other organs. In particular, the authors found that phosphatidylserine liposomes showed a higher affinity for liver hepatocyte uptake and much lower uptake in the spleen.

Compounds that interfere with ER glycosylation have been shown to inhibit infectivity of viruses such as hepatitis. One such compound is N-butyldeoxynojirimycin (“NB-DNJ”). However, despite a high inhibitory value in vitro, this compound required relatively large amounts to achieve its effect in tissue culture.

Liposomes containing a lipid prodrug have been used as drug systems for viral retinitis (Cheng et al., Invest Ophthalmol Vis Sci, May 2000, 41(6): 1523-1532).

Surface modified liposomes were shown to display anti-HIV activity (Kamps et al., Biochim. Biophys. Acta, 1278(1996): 183-190).

### SUMMARY OF THE INVENTION

The present invention relates to the use of pH-sensitive liposomes to deliver biologically active compounds. One embodiment is a pH-sensitive liposome comprising a biologically active compound that acts upon or within the endoplasmic reticulum.

Another embodiment is a method of using a pH-sensitive liposome comprising a biologically active compound to treat a viral infection.

Another embodiment is a method of using a pH-sensitive lip 6 some comprising a biologically active compound that acts upon or within the endoplasmic reticulum to treat pigmentation diseases.

Another embodiment is a method of preparing a pH-sensitive liposome comprising a compound that modifies the skin color, which could be included in a cosmetic composition.

### BRIEF DESCRIPTION OF THE DRAWINGS

FIG. 1 shows the effect of N-butyldeoxynojirimycin (“NB-DNJ”) encapsulated in different types of liposomes on tyrosinase activity in B16-F1 mouse melanoma cells. B16-F1 cells were cultured for 72 h in the absence (B16) or presence of 0.5 mM free NB-DNJ (B16+) and liposomes-entrapped inhibitor, respectively (see Table 1 below). Cell lysates were tested for DOPA-oxidase activity.

FIG. 2 shows reversibility of tyrosinase inhibition with liposome included NB-DNJ. Cells were incubated for 72 h in the presence of free and encapsulated NB-DNJ. NB-DNJ was, washed out of the cultures, and the cells were cultured for the times indicated.

FIG. 3 shows viability of B16-F1 mouse melanoma cells as a function of liposomal lipids concentration. B16-F1 cells were cultured for 72 h in the presence of 0.5 mM NB-DNJ encapsulated in pH-sensitive liposomes (M+L). Cells viability was determined by using the trypan blue staining test and their status was visualized by direct microscopic observation.

FIG. 4 shows tyrosinase activity in B16-F1 mouse melanoma cells incubated with empty pH-sensitive liposomes. DOPA-oxidase activity of cells treated with “empty” liposomes has been compared with the untreated B16-F1 cells (M) to test the cytotoxicity of liposomal lipids.

FIG. 5 shows DOPA-oxidase activity in cells treated with different concentrations of NB-DNJ included in pH-sensitive liposomes (M+L). Crude lysates of cells incubated for 72 h in the presence of different dilutions of the inhibitor were assayed for DOPA-oxidase activity.

FIG. 6 shows the effect of imino-sugars on mouse melanoma cells. The liposome included imino- sugars DNJ, NB-DNJ and,nonyl-DNJ have been added to the melanoma cells and the cells have been incubated for 72 h. The crude lysates have been assayed for tyrosinase activity.

FIG. 7 shows pigmentation of mouse melanoma cells in the presence of NB-DNJ included in liposomes. Cells were treated with for 72 h in the presence and absence of NB-DNJ free or included in liposomes, transferred into a microtiter plate and pelleted by centrifugation.

### DETAILED DESCRIPTION OF THE PREFERRED EMBODIMENTS

Unless otherwise specified, the words “a” or “an” mean “one or more”.

The term “treating” means preventing, reducing or ameliorating the symptoms of a disease or a condition that could lead to a disease.

One embodiment is a pH-sensitive liposome comprising a biologically active compound that acts upon or within the endoplasmic reticulum (“ER”). A biologically active compound may be included in the liposome as a single active agent or in combination with other biologically active compounds. The biologically active compound is preferably an anti-viral compound or a combination of biologically active compounds useful for treating a disease, preferably a viral disease, and more preferably hepatitis. A preferred type of anti-viral compound is a compound that inhibits ER N-glycosylation processing. A particularly preferred biologically active compound is N-butyldeoxynojirimycin (“NB-DNJ”).

The pH-sensitive liposome preferably comprises dioleoylphosphatidylethanolamine (“DOPE”) and cholesteryl hemisuccinate (“CHEMS”).

Another embodiment is a method of sing a pH-sensitive liposome comprising a biologically active compound to treat a viral infection. Preferably, the biologically active compound is a compound that acts upon or within the endoplasmic reticulum. More preferably, the biologically active compound is an anti-viral compound. The method preferably uses a pH-sensitive liposome which reduces the amount of anti-viral compound needed to achieve the same effect as a method which does not use such liposomes by a factor of at least 100, more preferably at least 500 and most preferably at least 1000.

Another embodiment is a method of using a pH-sensitive liposome comprising a biologically active compound that acts upon or within the endoplasmic reticulum to treat pigmentation diseases. Another embodiment is a method of preparing a pH-sensitive liposome comprising a compound that modifies skin color, which can be included in a cosmetic composition.

The following abbreviations are used to refer to the indicated items:

“M” refers to B16-F1 mouse melanoma cells;

“M+” refers to B16-F1 mouse melanoma cells incubated in the presence of 0.5 mM free NB-DNJ;

“M+L” refers to B16-P1 mouse melanoma cells incubated in the presence of 0.5 mM NB-DNJ included in pH-sensitive liposomes;

“M+L₀” refers to B16-F1 mouse melanoma cells incubated in the presence of “empty” pH-sensitive liposomes;

“CHEMS” refers to cholesteryl hemisuccinate;

“Chol” refers to cholesterol;

“DOPA” refers to β-3,4-dihydroxyphenylalanine;

“DOPE” refers to dioleoylphosphatidylethanoiamine;

“EDTA” refers to ethylene diaminetetraacetic acid disodium salt;

“ER” refers to endoplasmic reticulum;

“FCS” refers to fetal calf serum;

“MLV” refers to multi-lamellar vesicle;

“NB-DNJ” refers to N-butyldeoxynojirimycin;

“PA” refers to phosphatidic acid;

“PBS” refers to phosphate-buffered saline;

“PC” refers to phosphatidylcholine;

“PE” refers to phosphatidylethanolamine;

“PS” refers to phosphatidyl serine; and

“SUV” refers to small unilamellar vesicle:

The present invention is further illustrated by, though in no way limited to, the following examples.

1. Effect of pH-Sensitive Liposomes Comprising NB-DNJ

To monitor the efficiency of encapsulated NB-DNJ delivery in the cytoplasm of mammalian cells, B16 mouse melanoma cells were cultivated in the presence of various lipid formulations and the modifications in the activity of tyrosinase synthesized in these cells was assayed. Encapsulation of NB-DNJ in pH-sensitive liposomes reduces the required dose of NB-DNJ by a factor of 1000.

2. Materials and Methods

2.1. Materials

Lipids for liposome preparation—dioleoylphosphatidylethanolamine (DOPE), cholesterylhemisuccinate (CHEMS), phosphatidylethanolamine (PE), phosphatidylcholine (PC), phosphatidylserine (PS) and cholesterol (Chol) were purchased from Sigma Chemical Co. (St. Louis, Mo., and U.S.A.). All other chemicals were analytical grade or the best grade available.

2.2. Cell Culture

B16-F1 mouse melanoma cells (European Collection of Animal Cell Culture, Porton Down, U.K.) were cultured in RPMI 1640 medium (Life Technologies, Paisley, Scotland, and U.K.) as described in ref. [8]. Cells were incubated with liposomes for 72 h at 37° C. and their viability was determined by using the trypan blue staining test. Following the incubation time, cells were harvested, washed three times with PBS and lysed in 10 mM phosphate buffer, pH 6.8 containing 1% Nonidet P40 and proteinase inhibitors.

2.3. Preparation of Liposomes

Liposomes were made according to the general method for small unilamellar vesicles (SUV) preparation from multi-lamellar vesicles (MLV) (ref. [15]). In particular, a mixture of the appropriate amounts of phospholipids from stock solutions in chloroform/methanol (95:5) was dried in a rotary evaporator under reduced pressure. Dried lipids were hydrated with PBS containing different concentration of the inhibitor to be encapsulated. Resulting MLV were sonicated in a bath-type sonicator (at least 1 h, at room temperature) followed by a probe sonicator (about 10 min, with intervals, cooled in ice/water) to form a stable emulsion. After sonication, metal particles from the probe and large MLV were removed by centrifugation (15 min, 12 000 rpm, 4° C.). The diameter of SUV liposomes, as shown by transmission electron microscopy varied between 50 and 200 nm. Liposomal formulations were filtered under sterile conditions through a 0.22 μm filter (Milipore). In some experiments, liposomes were separated from free NB-DNJ by ultracentrifugation at 159,000 g for 4 h. The amount of encapsulated NB-DNJ was monitored with ¹⁴C labelled NB-DNJ. The inclusion yield was 1-5 % as calculated by experiments using labelled NB-DNJ (ref. [16]).

2.4. Tyrosinase Activity

Tyrosinase activity was measured by the DOPA oxidase assay that measures the conversion of DOPA to DOPAchrome via DOPA quinone. Briefly, 50 μl of cell lysate was mixed with 950 μl of 1 mM DOPA in 0.1 M sodium phosphate buffer, pH 7.2 at 37° C. and the reaction was followed spectrophotometrically at 475 nm by the chromogenic appearance of DOPAchroine from 1 mM DOPA (ref. [8]).

3. Results

To investigate the uptake of liposome encapsulated NB-DNJ in living cells,.the activity of tyrosinase was monitored, a constitutively expressed enzyme of melanoma cells, highly sensitive to small variations of the NB-DNJ concentration in the culture medium (ref. [8]). Inhibition of N-glycosylation processing in the presence of NB-DNJ yields a tyrosinase polypeptide caring Glc3Man7-9GlcNAc glycans. As a result, the interaction with the ER chaperone calnexin is abolished and tyrosinase folding escapes the ER quality control resulting in an enzyme devoided of its biological activity (ref. [7]). A dramatic decrease of tyrosinase activity was observed when B16 mouse melanoma cells were cultivated in the presence of concentrations varying fom 0.5 mM-5 mM NB-DNJ (ref. [6]), recommending tyrosinase as a sensitive probe to monitor the intracellular delivery of liposome encapsulated NB-DNJ.

Five lipid compositions were tested generating two types of liposomes, pH sensitive (s) and pH insensitive (i): s₁, consisting of phosphatidylethanolamine (PE) and cholesterylhemisuccinate (CHEMS); s₂, made of dioleoylphosphatidylethanolamirie (DOPE) and CHEMS; i₁, consisting of phosphatidylcholine (PC) and cholesterol (Chol); i₂, prepared from PC, Chol and phosphatidylserine (PS); i₃, containing PC and CHEMS (Table 1).

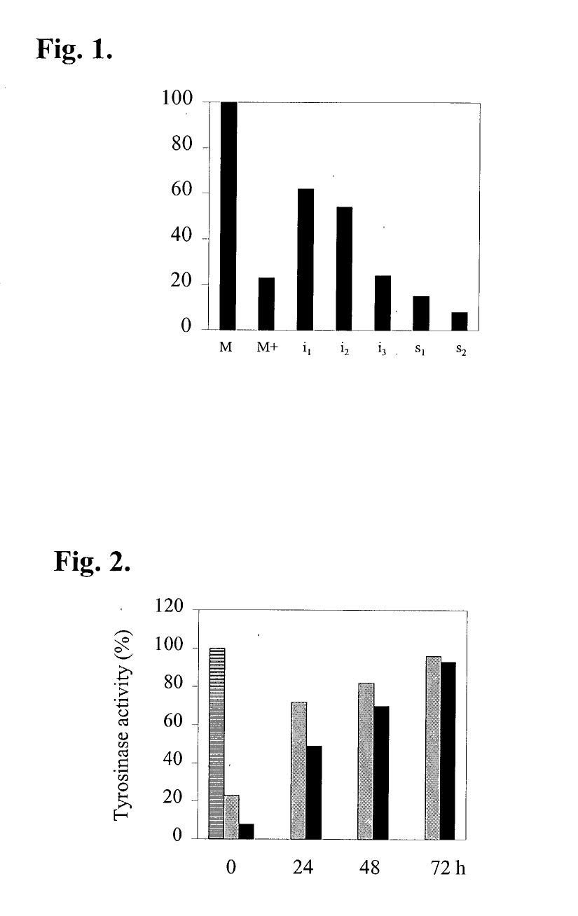

Liposomes prepared in 0.5 mM NB-DNJ were added to the culture medium and B16-F1 mouse melanoma cells were cultivated at 37° C. in 5% CO₂ for 72 h. As shown in FIG. 1 when cells were cultured in the presence of NB-DNJ included in pH-insensitive liposomes, tyrosinase activity was decreased to 62% for PC/Chol liposomes (i₁) and to 51% in the case of PC/Chol/PS liposomes-entrapped inhibitor (i₂). Although the level of tyrosinase inhibition went down to 25% for NB-DNJ included in PC/CHEMS liposomes (i3), any of the three pH insensitive liposomes formulations tested was able to produce the inhibition level obtained in the presence of free NB-DNJ (FIG. 1).

Similar experiments were performed with different formulations of pH-sensitive liposomes. Tyrosinase activity decreased by 85% in cells incubated in the presence of pH-sensitive PE/CHEMS liposomes including NB-DNJ (s₁). When cells were cultivated in the presence of pH-sensitive liposomes composed of DOPE/CHEMS (S₂) tyrosinase activity decreased by 92%, recommending this liposomal formulation as the most efficient in the delivery of the glycosylation inhibitor. Similar results have been obtained with liposomes purified by ultracentrifugation. Usually, pH-insensitive liposomes are taken up by endocytosis, which limits the efficient delivery of intact encapsulated material to the cytosol. A partial delivery of NB-DNJ into the cytoplasm could explain the ˜50 % inhibition of tyrosinase obtained with these liposomes as compared with the 98% inhibition resulted following the treatment of the B16-F1 cells with the drug included in pH-sensitive liposomes.

CHEMS included in the composition of either pH sensitive or pH insensitive liposomes confers a remarkable property of efficient cytoplasmic delivery, possibly due to its negative charge that facilitates their penetration through the plasma membrane. The higher efficiency observed when CHEMS was used in conjunction with DOPE, in pH-sensitive liposomes, could be explained by the destabilization of pH sensitive liposomes due to the protonation of CHEMS in the acidic endosomes, resulting in the release of the-imino sugars in the cytoplasm. Total reversibility of tyrosinase activity was observed when NB-DNJ containing liposomes were washed out of the culture (FIG. 2), confirming that liposome included NB-DNJ induces reversible modifications in the cell, hence reinforcing the potential use of this iminosugar as a potential therapeutic drugs. Recovery of tyrosinase activity is slower in liposome treated cells as compared to free drug treated cells (50% recovery in 24 h time as compared to 75% recovery for free NB-DNJ).

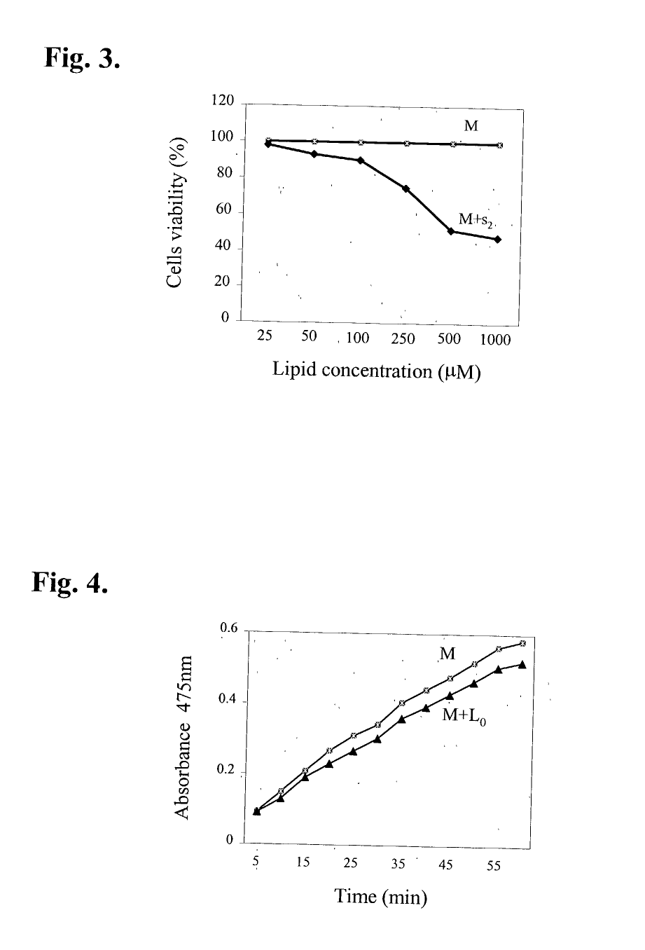

To ensure that liposomes do not induce side effects to the cultured mammalian cells, their cytotoxicity was evaluated. The effect of a preferred lipid formulation (pH-sensitive lihposomes DOPE/CHEMS) was evaluated using various lipid concentrations (25-1000 μM). The results in FIG. 3 show that cell viability was affected at high liposomal lipid concentration. The cytotoxicity gradually-appeared following the incubation in the presence of 250 μM lipid concentration and became significant after a 500 μM lipid concentration. It was established that a preferred lipid concentration in the culture medium that allows a cell viability of more than 90%/p was 100 μM. This lipid concentration was used in all liposomes formulations.

As a second test, tyrosinase activity was monitored in B16-F1 cells incubated with “empty” pH-sensitive liposomes in identical conditions. As shown in FIG. 4, tyrosinase enzymatic activity in B16-F1 cells is not affected by empty pH-sensitive liposomes.

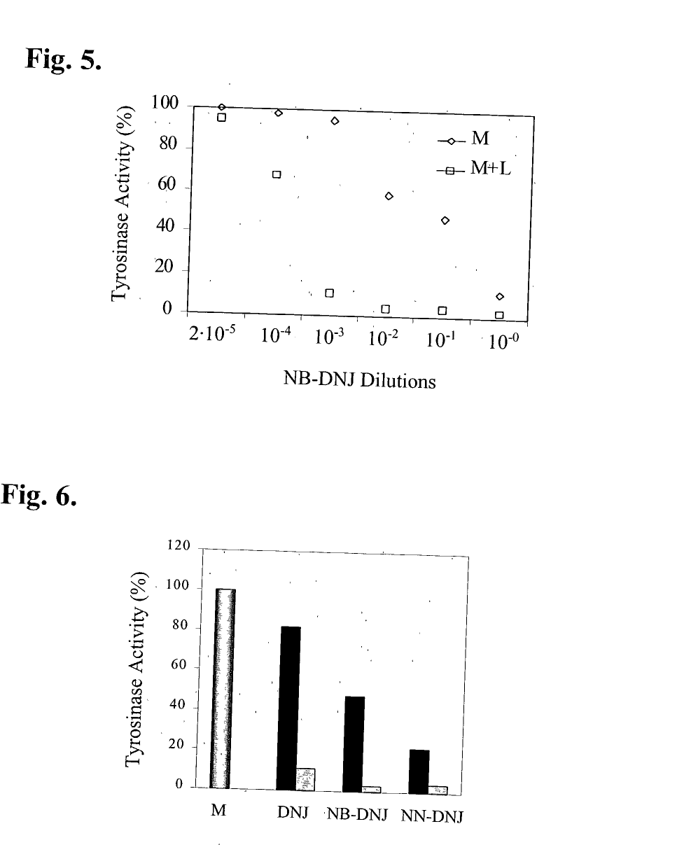

To evaluate the pH-sensitive liposomes (DOPE/CHEMS 6:4) efficiency in delivering reduced amounts of inhibitor that inactivate tyrosinase, several concentrations of NB-DNJ were tested. B16-F1 cells were incubated in the presence of different amounts of NB-DNJ included in pH-sensitive liposomes and tyrosinase activity was assayed. The amount of the inhibitor included in pH-sensitive liposomes required for the ER inactivation of tyrosinase was about 100-1000 times lower than the amount of free NB-DNJ added in the culture medium (FIG. 5). Higher dilutions of AB-DNJ included in liposomes had no more effect on tyrosinase enzymatic activity (FIG. 5).

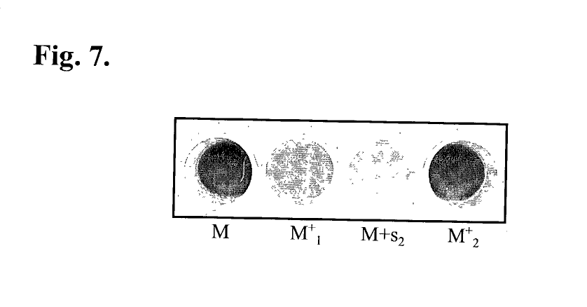

Similar results were obtained when two other iminosugars, deoxynojirimycin (DNJ) and nonyl-deoxynojirimycin (nonyl-DNJ) have been entrapped in DOPE/CHEMS liposomes. As shown in FIG. 6, tyrosinase activity was dramatically decreased when cells were treated with liposome included drugs as compared to the free compounds. These results show that the liposome formulations used in this study could be used for inclusion of various glycosylation inhibitors, with highly efficient intracellular delivery and displaying high stability in serum containing media.

Tyrosinase is the regulatory enzyme of melanogenesis in melanocytes. Therefore, inactivation of tyrosinase is associated with lack of pigmentation due to the absence of melanin synthesis in specialized cells. To correlate the results on tyrosinase activity, with the melanin content, the pigmentation of the melanoma cells treated With liposomes was observed. Visual inspection of untreated melanoma cells showed a normal level of pigmentation, whereas cells treated with 0.5 mM free NB-DNJ and 0.5 μM liposome entrapped NB-DNJ, respectively, showed a similar dramatic decrease in their pigmentation (FIG. 7). The lack of pigmentation in melanoma cells treated with minute amounts of liposomal NB-DNJ correlates well with the abolishment of tyrosinase activity as a result of N-glycosylation processing inhibition, reinforcing the efficiency of the cytoplasmic delivery of the drug encapsulated in liposomes.

Thus, the present invention provides a liposomal system that offers a direct, effective and selective delivery of biologically active materials into cells. While the invention is not bound by its theory of operation, it is probable that liposomes adsorb to the cell surface by a mechanism that depends on liposome negative charge and are internalized into the cell, through the endocytic pathway. NB-DNJ has been shown to be effective in retaining the hepatitis B virus inside the hepatic cells, thus preventing the multiplication of the virus. Taking into account the potential use of NB-DNJ in treating the hepatitis B, the encapsulation of the drug in liposomes could be used to target small and less toxic NB-DNJ concentration to the cytoplasm and into the ER of the hepatic cells.

References

1. Komfeld, R. and Komfeld, S. (1985) Annu.Rev.Buiochem. 54, 631-664.

2. Varki, A. (1993) Glycobiology 3, 97-13.

3. Metha, A., Lu, X., Block, T., Blumberg, B. and Dwek, R. A. (1997) Proc. Natl. Acad. Sci. U S A 94, 3721-3728.

4. Block, T. M., Lu, X. Y., Mehta, A. S., Blumberg, B. S., Tennant, B., Ebling, M., Korba, B., Lansky, D. M., Jacob, G. S., Dwek, R. A., (1998) Nature Med. 4, 610-614.

5. Branza-Nichita, N., Durantel, D., Carrouee-Durantel, S., Dwek; R. A., Zitzmann, N. (2001) J.Virol. 75, 3527-3536.

6. Ptrescu, S. M., Branza-Nichita, N., Negroiu, G., Petrescu, A-J., Dwek, R. A (2000). Biochem. 39, 5229-5237.

7. Branza-Nichita, N., Petrescu, A-J., Dwek, R. A, Wormald, M., Platt, F. M., Petresu, S. M. (1999) Biochem.Biophys.Res.Commun. 261, 720-725.

8. Petrescu, S. M., Petrescu, A. J., Titu, H. N., Dwek, R. A. and Platt, F. M. (1997) J. Biol. Chem. 272, 15796-15803.

9. Butters, T. D., Dwek, R. A., Platt, F. M. (2000) Chem.Rev.100, 4863-4696

10. Branza-Nichita, N., Petrescu, A. J., Negroiu, G., Dwek, R. A., Petrescu, S. M. (2000), Chem.Rev. 100, 4697-4711.

11. Karlsson, G., Butters, T., Dwek, R. A. and Platt, F. M. (1993) J. Biol. Chem. 268, 570-576.

12. Straubinger, R. M. (1993) Methods Enzymol. 221, 361-376.

13. Alving, C. R., Velinova, M., Regts, J. and Scherpmof, G. L. (1997) Hepatology 26, 416-422.

14. Lee, K.-D., Oh, Y.-K., Portnoy, D. A. and Swanson, J. A.- (1996) J. Biol. Chem. 271, 7249-7252.

15. Gregoriadis, G., Liposome Preparation and Related Techniques in Liposome Technology, 2nd edition, CRC Press, Boca Raton, Fla. 1993

TABLE 1
Liposome formulations used in this paper

| Liposome type | Lipid composition | Lipid molar ratio |
|---|---|---|
| pH-sensitive (s₁) | PE:CHEMS | 3:2 |
| pH-sensitive (s₂) | DOPE:CHEMS | 3:2 |
| pH-insensitive (i₁) | PC:Chol | 3:1 |
| pH-insensitive (i₂) | PC:Chol:PS | 7:2:1 |
| pH-insensitive (i₃) | PC:CHEMS | 3:1 |

It will be apparent to those skilled in the art that various modifications and variations can be made to the compositions, methods, and processes of this invention. Thus, it is intended that the present invention cover such modifications and variations, provided they come within the scope of the appended claims and their equivalents.

The disclosure of all publications cited above are expressly incorporated herein by reference in their entireties to the same extent as if each were incorporated by reference individually.

What is claimed is:

1. A pH-sensitive liposome comprising a biologically active compound that acts upon or within the endoplasmic reticulum.

2. The liposome of claim 1, further comprising one or more additional biologically active compounds.

3. The liposome of claim 1, wherein the biologically active compound that acts upon or within the endoplasmic reticulum is a compound that interferes with glycosylation.

4. The liposome of claim 3, wherein the biologically active compound that interferes with glycosylation is N-butyldeoxynojirimycin.

5. The liposome of claim 4, wherein the liposome comprises dioleoylphosphatidylethanolamine and cholesteryl hemisuccinate.

6. The liposome of claim 1, wherein the liposome comprises dioleoylphosphatidylethanolamine and cholesteryl hemisuccinate.

7. A method of treating a viral infection comprising administering to a subject in need thereof a therapeutically effective amount of the liposome of claim 1.

8. A method of treating a viral infection comprising administering to a subject in need thereof a therapeutically effective amount of the liposome of claim 5.

9. A pH-sensitive liposome comprising a compound that modifies skin color.

10. The liposome of claim 9, wherein the liposome comprises dioleoylphosphatidylethanolamine and cholesteryl hemisuccinate.

11. A cosmetic composition comprising the liposome of claim 9.

12. A method of modifying skin color, comprising administering to a subject in need thereof an effective amount of the liposome of claim 9.

13. A pH-sensitive liposome comprising a compound for treating a pigmentation disease.

14. The liposome of claim 13, wherein the liposome comprises dioleoylphosphatidylethanolamine and cholesteryl hemisuccinate.

15. A method of treating a pigmentation disease, comprising administering to a subject in need thereof an effective amount of the liposome of claim 13.
